(19) United States
(12) Patent Application Publication
(10) Pub. No.: US 2023/0065000 A1
(43) Pub. Date: Mar. 2, 2023

(54) SYSTEM AND METHOD FOR MONITORING AND ANALYZING IMPACT DATA TO DETERMINE CONSCIOUSNESS LEVEL AND INJURIES

(71) Applicant: ANIRUDHA SURABHI VENKATA JAGANNADHA RAO, Dallas, TX (US)

(72) Inventor: ANIRUDHA SURABHI VENKATA JAGANNADHA RAO, Dallas, TX (US)

(21) Appl. No.: 17/983,649

(22) Filed: Nov. 9, 2022

Related U.S. Application Data
(63) Continuation-in-part of application No. 17/201,112, filed on Mar. 15, 2021.

(51) Int. Cl. A61B 5/00; A61B 5/11

(52) U.S. Cl. CPC A61B 5/7282; A61B 5/0022; A61B 5/1114; A61B 5/1112; A61B 5/7264; A61B 5/1128; A61B 5/747; A61B 5/4064; A61B 5/6803; A61B 2562/0219; A61B 2503/22; A61B 2560/0475

(57) ABSTRACT

A system for monitoring and analyzing impact data to determine consciousness level and injuries, comprising: impact event monitoring device monitors impact event that occurs to first end-user, impact event monitoring device integrated into objects; impact event monitoring device collects impact data of objects, first end-user using sensors, impact sensing unit, motion detection unit, GPS module, image capturing unit; network module send impact data to impact event monitoring and analyzing module, impact event monitoring and analyzing module convert impact data into Yaw, Pitch and Roll data, impact event monitoring and analyzing module analyze Yaw, Pitch and Roll data with machine learning techniques and deep learning techniques; impact event monitoring and analyzing module report and send emergency notifications to second computing device, impact data accessing module enable second end-user to examine location and intensity of impact event to understand consciousness level and injuries for providing better treatment to first end-user.

### CROSS-REFERENCE TO RELATED APPLICATIONS

This patent application claims priority benefit of U.S. Provisional Patent Application No. 62/990,456, entitled “System and Method for Monitoring, Identifying and Reporting Impact Events in Real-Time”, filed on 17 Mar. 2020, and is a continuation-in-part of U.S. Non-Provisional patent application Ser. No. 17/201,112 entitled “System and Method for Monitoring, Identifying and Reporting Impact Events in Real-Time”, filed on 15 Mar. 2021. The entire contents of both the patent applications are hereby incorporated by reference herein in its entirety.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter relates generally to an emergency event management system. More particularly, the present disclosure relates to a system and method for analyzing impact data to determine consciousness level and injuries that occur to objects/subjects in real-time.

### BACKGROUND

Generally, the participation of athletes in athletic activities is increasing at all age levels. All participants are potentially exposed to physical harm as a result of such participation. Physical harm is more likely to occur in athletic events where collisions between participants frequently occur (e.g., football, field hockey, lacrosse, ice hockey, soccer, and so forth). In connection with sports such as football, hockey, and lacrosse, where deliberate collisions between participants occur, the potential for physical harm and/or injury is greatly enhanced.

For example, in the world, each year, there are a million athletes with age below twenty-four who play contact sports such as football, basketball, hockey, soccer, boxing, and mixed martial arts (mixed martial arts (MMA)). All these young athletes are at risk for head injury with a concussion (concussive traumatic brain injuries (CTBI)) and long-term brain dysfunction due to repeated head impacts. These young athletes with developing neurological systems suffer a large part of the 3.8 million CTBIs that occur annually and are at high risk of developing long-term adverse neurological, physiological, and cognitive deficits. The conditions of head impacts responsible for CTBI and potential long-term deficits in athletes are unknown. Head injuries are caused by positive and negative acceleration forces experienced by the brain and may result from linear or rotational accelerations (or both). Both linear and rotational accelerations are likely to be encountered by the head at impact, damaging neural and vascular elements of the brain. Similarly, the percentage of vehicular crashes both on-road and off-road has been increasing rapidly all over the world. In most cases, the information on the state of the vehicle during the incident is not known. Many of the deaths and permanent injuries could have been prevented if the emergency responders had arrived more quickly. Too many precious minutes are lost because calls for help are delayed or because emergency responders cannot quickly locate the accident.

In comparison to an automobile, one is exposed to considerably higher risk in road traffic as a motorbike rider like, for example, a motorcycle rider and/or a motor-trike rider and/or a quad rider and/or a scooter rider, and/or a moped rider. Among other things, this is because of the different driving physics and the constantly unstable state of balance, as well as the particular physical and psychological stress when riding a motorbike and the limited field of view. At the same time, motorbike riders are considerably more sensitive to weather factors and other disturbance factors like, for instance, poor road conditions or unexpected traffic conditions. In addition, there is no protective body shell in the case of a motorbike. Despite protective clothing, motorbike riders are unprotected road users during collisions or crashes due to the lack of passive safety. Hence, there is a need for a system to sense the impact events, determine level of consciousness, location of the objects/subjects and intensity of each impact, thereby activating the emergency protocol examiners to properly diagnose the extent and severity of the injury for providing better treatment to head/brain injuries.

In the light of the aforementioned discussion, there exists a need for a system with novel methodologies that would overcome the above-mentioned challenges.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards a system and method for analyzing impact data to determine consciousness level and injuries that occur to objects and/or subjects in real-time.

An objective of the present disclosure is directed towards a system that determines a location of the objects and/or subjects and the intensity of an impact event.

An objective of the present disclosure is directed towards a system that provides a wirelessly linked impact, anomaly sensing, and reporting system.

Another objective of the present disclosure is directed towards a system that activates an emergency protocol automatically by an impact event monitoring and analyzing module.

Another objective of the present disclosure is directed towards a system that delivers the impact data to medical examiners to properly diagnose the extent and severity of the injuries to provide better treatment for head/brain injuries.

Another objective of the present disclosure is directed towards a system that identifies the accurate positions of the head and/or body position of the subject and the geographical location of the object and/or subject at the time of the impact event and are analyzed by medical professionals to gauge the extent of the injury.

Another objective of the present disclosure is directed towards a system that determines the mental state, consciousness, alertness of the objects and subject after a crash or impact using a plurality of motion sensors.

Another objective of the present disclosure is directed towards a system that relates to a safety system for a helmet/wearable, as well as a corresponding method for triggering a safety alert.

Another objective of the present disclosure is directed towards a system that includes an impact event monitoring device or a driver/rider assistance device integrated into the helmets for use in the safety and transport industry/vehicle industry (mental state of a rider/worker/person wearing a helmet).

Another objective of the present disclosure is directed towards a system that improves the safety of motorcycling.

Another objective of the present disclosure is directed towards a system that monitors a predefined pattern of head movements of a first end-user, which are categorized as dangerous riding.

Another objective of the present disclosure is directed towards a system that detects if the first end-user deviates from the predefined posture for longer than a predetermined period of time and transmits a signal to a second computing device using an algorithm embedded in a processing device.

Another objective of the present disclosure is directed towards a system that relates to machine learning and deep learning techniques for the analysis of the movement behavior of the first end-user based on a combination of accurate positions and locations and impact data obtained from the first set of sensors, the second set of sensors, and the third set of sensors along with the first computing device.

Another objective of the present disclosure is directed towards a system that determines the level of consciousness of the first end-user after experiencing the crash/impact on the head/helmet.

According to an exemplary aspect, a system includes an impact event monitoring device configured to monitor an impact event that occurs to a first end-user, the impact event monitoring device is integrated into one or more objects to detect the impact event.

According to another exemplary aspect, the impact event monitoring device comprises a processing device configured to collect impact data of at least one of: the objects; and the first end-user; using a first set of sensors, a second set of sensors, and a third set of sensors, an impact sensing unit, a motion detection unit, a GPS module, and an image capturing unit upon detecting the impact event, the impact data comprises one or more accurate positions and locations and sensor data.

According to another exemplary aspect, a network module configured to send the impact data to an impact event monitoring and analyzing module, the impact event monitoring and analyzing module configured to receive impact data from the impact event monitoring device and convert the impact data into Yaw, Pitch and Roll data, wherein the Yaw, Pitch and Roll data are respective rotations of the first end-user around X, Y and Z axes and the Yaw, Pitch and Roll data are used to track one or more head movements of the first end-user, the impact event monitoring and analyzing module also configured to analyze the Yaw, Pitch and Roll data with one or more machine learning techniques and one or more deep learning techniques to determine consciousness level and injuries that occur to the first end-user in the impact event.

According to another exemplary aspect, the impact event monitoring and analyzing module configured to report the impact event and send emergency notifications along with the impact data to a second computing device over a network, the second computing device comprises an impact data accessing module configured to enable a second end-user to examine location and intensity of the impact event to understand the consciousness level and the injuries for providing better treatment to the first end-user.

### BRIEF DESCRIPTION OF THE DRAWINGS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

FIG. 1 is a block diagram depicting a schematic representation of a system for monitoring and analyzing impact data to determine consciousness level and injuries that occur to objects and/or subjects in real-time, in accordance with one or more exemplary embodiments.

FIG. 2 is a block diagram depicting an impact event monitoring device 102 shown in FIG. 1, in accordance with one or more exemplary embodiments.

FIG. 3 is a block diagram depicting a schematic representation of the impact event monitoring and analyzing module 114 shown in FIG. 1, in accordance with one or more exemplary embodiments.

FIG. 4 is a flowchart depicting an exemplary method of reporting impact events to the second end-users, in accordance with one or more exemplary embodiments.

FIG. 5 is a flowchart depicting an exemplary method of tracking the objects using an impact event monitoring device, in accordance with one or more exemplary embodiments.

FIG. 6 is a flowchart depicting an exemplary method of displaying the activity recognition and performance grading of the objects, in accordance with one or more exemplary embodiments.

FIG. 7 is a flowchart depicting an exemplary method of displaying the movements of objects/subjects during the crash, in accordance with one or more exemplary embodiments.

FIG. 8 is a flowchart depicting an exemplary method for analyzing impact events and determining the consciousness of subjects after the crash, in accordance with one or more exemplary embodiments.

FIG. 9 is a flowchart depicting another exemplary method for analyzing impact events and determining the consciousness of subjects after the crash, in accordance with one or more exemplary embodiments.

FIG. 10 is a block diagram illustrating the details of a digital processing system in which various aspects of the present disclosure are operative by execution of appropriate software instructions.

Furthermore, the objects and advantages of this invention will become apparent from the following description and the accompanying annexed drawings.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and so forth, herein do not denote any order, quantity, or importance, but rather are used to distinguish one element from another.

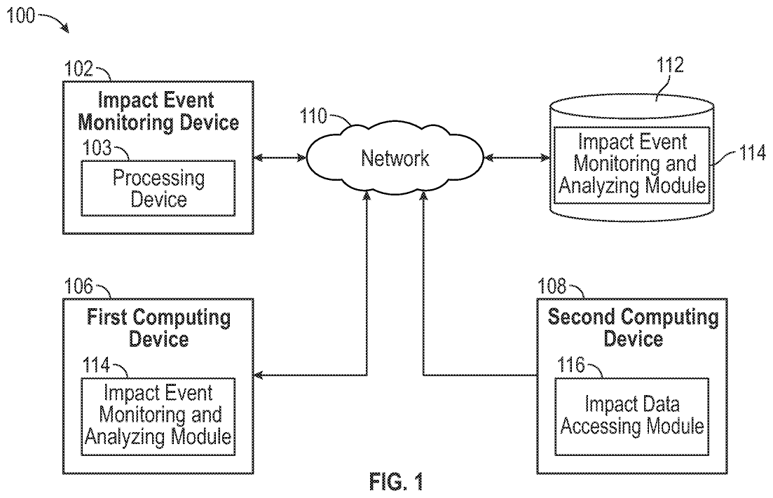

Referring to FIG. 1 is a block diagram 100 depicting a schematic representation of a system for monitoring and analyzing impact data to determine consciousness level and injuries that occur to objects and/or subjects in real-time, in accordance with one or more exemplary embodiments. The objects may include, but not limited to, vehicles, car seats, wristbands, helmets, headbands, and so forth. The subject may be a first end-user. The first end-user may include, but not limited to, a driver, an athlete, a motorist, a passenger, a vehicle owner, a vehicle user, an individual, a rider, and so forth.

The system 100 includes an impact event monitoring device 102, a processing device 103, a first computing device 106, a second computing device 108, a network 110, a central database 112 and an impact event monitoring and analyzing module 114, and an impact data accessing module 116. The system 100 may include multiple impact event monitoring devices 102, multiple processing devices 103, and multiple computing devices 106, 108. The system 100 may link multiple impact event monitoring devices 102, multiple processing devices 103, and multiple computing devices 106, 108 into a single hub that may display devices information at a glance.

The impact event monitoring device 102 may be an inertial measurement unit. The impact event monitoring device 102 may be configured to detect and track an object's motion in three-dimensional space and allows the first end-users to interact with the first computing device 106 by tracking motion in free space and delivering these motions as input commands. The impact event monitoring device 102 may be integrated into a vehicle, steering wheel, dashboard, car seats (if the user does not require an image capturing unit), headbands, helmets, electronic devices, and so forth. The impact event monitoring device 102 may be configured to detect/sense the impact events, and emergency events, occur to the objects and/or subjects. The impact events may include, but not limited to, non-accidental emergency events relating to the vehicle (e.g., a theft of the vehicle), or emergency events relating specifically to the occupant(s) of the vehicle (e.g., a medical impairment of an occupant of the vehicle, regular events in the course of rough activity, a kidnapping or assault of an occupant of the vehicle, etc.), accidental emergency events relating to vehicle or other transport crashes, fires, medical emergencies, or other threats to safety, movements and motion, injuries, abnormalities, interrupts, impacts or anomalies and so forth. The impact event monitoring device 102 may be configured to obtain the impact data of the impact event that occur to the objects, and/or the first end-user and reports the impact data to the second computing device 108 over the network 110. The impact data includes one or more accurate positions and locations and the sensor data. The accurate positions and locations may include, but not limited to, a head and/or body position of the subject, a geographical position of the object, a geographical position of the subject and so forth. The sensor data may include, but not limited to, quaternions, Euler angles, vital statistics, the rotational angle of the head of the individual or the object, movement and/or motion of the individual or the object, location acceleration and gyroscope vectors, velocity, location and so forth.

The network 110 may include but not limited to, an Internet of things (IoT network devices), an Ethernet, a wireless local area network (WLAN), or a wide area network (WAN), a Bluetooth low energy network, a ZigBee network, a WIFI communication network e.g., the wireless high speed internet, or a combination of networks, a cellular service such as a 4G (e.g., LTE, mobile WiMAX) or 5G cellular data service, a RFID module, a NFC module, wired cables, such as the world-wide-web based Internet, or other types of networks may include Transport Control Protocol/Internet Protocol (TCP/IP) or device addresses (e.g. network-based MAC addresses, or those provided in a proprietary networking protocol, such as Modbus TCP, or by using appropriate data feeds to obtain data from various web services, including retrieving XML data from an HTTP address, then traversing the XML for a particular node) and so forth without limiting the scope of the present disclosure. The impact event monitoring and analyzing module 114 may be configured to establish the communication between the impact event monitoring device 102 and the first computing device 106 through the network 110. The impact event monitoring device 102 may be configured to activate impact protocols (emergency protocols) to establish communication with the first computing device 106 and the second computing device 108 through the impact event monitoring and analyzing module 114 via the network 110.

The first computing device 106 and the second computing device 108 may be operatively coupled to each other through the network 110. The first and second computing devices 106 and 108 may include but not limited to, a computer workstation, an interactive kiosk, and a personal mobile computing device such as a digital assistant, a mobile phone, a laptop, and storage devices, backend servers hosting the database and other software, and so forth. The first computing device 106 may be operated by the first end-user. The second computing device 108 may be operated by the second end-user. The second end-user may include, but not limited to, medical professionals, a medical examiner(s), an emergency responder(s), an emergency authority medical practitioner(s), a doctor(s), a physician(s), a family member(s), a friend(s), a relative(s), a neighbor (s), an emergency service provider(s), and so forth.

Although the first and second computing devices 106, 108 are shown in FIG. 1, an embodiment of the system 100 may support any number of computing devices. Each computing device supported by the system 100 is realized as a computer-implemented or computer-based device having the hardware or firmware, software, and/or processing logic needed to carry out the intelligent messaging techniques and computer-implemented methodologies described in more detail herein.

The impact event monitoring and analyzing module 114, which is accessed as mobile applications, web applications, software that offers the functionality of accessing mobile applications, and viewing/processing of interactive pages, for example, are implemented in the first and second computing devices 106, 108 as will be apparent to one skilled in the relevant arts by reading the disclosure provided herein. The impact event monitoring and analyzing module 114 may be downloaded from the cloud server (not shown). For example, the impact event monitoring and analyzing module 114 may be any suitable applications downloaded from, GOOGLE PLAY® (for Google Android devices), Apple Inc.'s APP STORE® (for Apple devices, or any other suitable database). In some embodiments, the impact event monitoring and analyzing module 114 may be software, firmware, or hardware that is integrated into the first and second computing devices 106, 108, the central database 112 and the impact event monitoring device 102.

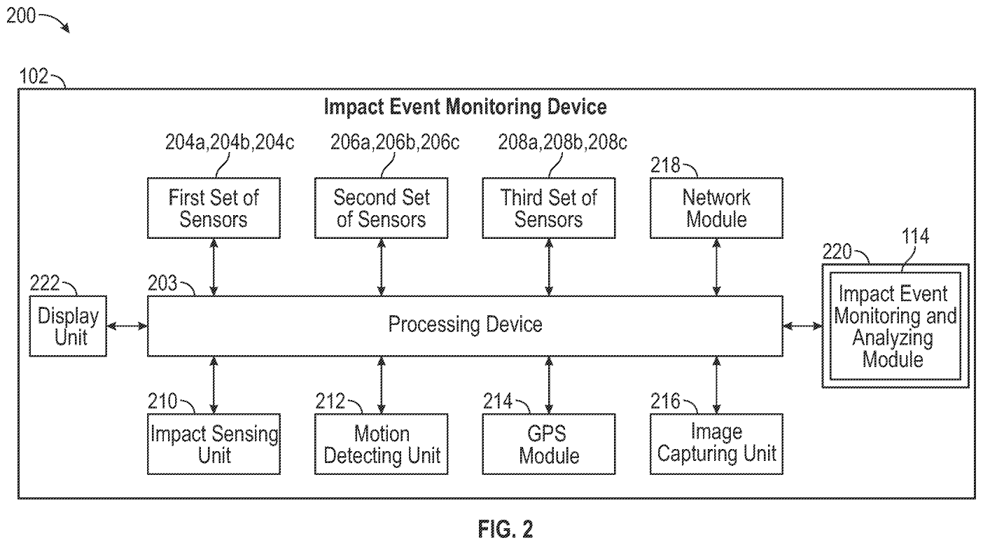

The processing device 103 may include, but not limited to, a microcontroller (for example ARM 7 or ARM 11), a raspberry pi3 or a Pine 64 or any other 64 bit processor which can run Linux OS, a microprocessor, a digital signal processor, a microcomputer, a field programmable gate array, a programmable logic device, a state machine or logic circuitry, Arduino board. A set of sensors (204a, 204b and 204c, 206a, 206b and 206c, 208a, 208b and 208c shown in FIG. 2) may be electrically coupled to the processing device 103.

According to an exemplary embodiment of the present disclosure, a system for monitoring, identifying and reporting impact events and analyzing impact data to determine consciousness level and injuries that occur to objects and/or subjects in real-time, includes the impact event monitoring device 102 configured to monitor impact events of objects and subjects through the processing device 103, the processing device 103 configured to identify impact data of the objects and the subjects using the first set of sensors 204a, 204b, and 204c, the second set of sensors 206a, 206b and 206c, and the third set of sensors 208a, 208b and 208c, the impact sensing unit 210, the motion detection unit 212, and the GPS module 214 (As shown in FIG. 2). The impact data includes one or more accurate positions and locations and the sensor data. The accurate positions and locations may include, but not limited to, a head and/or body position of the subject, a geographical position of the object, a geographical position of the subject and so forth. The sensor data may include, but not limited to, quaternions, Euler angles, vital statistics, the rotational angle of the head of the individual or the object, movement and/or motion of the individual or the object, location acceleration and gyroscope vectors, velocity, location and so forth.

The processing device 103 may be configured to enable the image capturing unit 216 (as shown in FIG. 2) to capture and record the objects and the subjects. The processing device 103 may include the memory unit 220 is programmed with the impact event monitoring and analyzing module 114.

The network module 218 (as shown in FIG. 2) may be configured to report the accurate positions and locations, the sensor data, and media files (impact data) of the impact events to the first computing device 106 and the second computing device 108. The media files of the impact event may include, but not limited to, images, pictures, videos, GIF's, and so forth. The impact event monitoring and analyzing module 114 may be configured to enable the first computing device 106 and the second computing device 108 to analyze the accurate positions and locations of the objects and the subjects to understand the extent of the impact events. The central database 112 may be configured to store the accurate positions and locations, the sensor data, and media files of the impact events.

According to another exemplary embodiment of the present disclosure, the system for monitoring, identifying and reporting impact events and analyzing impact data to determine consciousness level and injuries that occur to objects and/or subjects in real-time, comprising the impact event monitoring device 102 is configured activate the emergency protocols to establish communication with the first computing device 106 and the second computing device 108 over the network 110, the impact event monitoring device 102 is configured to report to report the impact event and deliver emergency notifications of the impact events that occur to the objects and subjects to the second computing device 108 over the network 110.

A method for monitoring, identifying and reporting impact events in real-time, comprising: monitoring objects and subjects by the event monitoring device 102; detecting accurate positions and locations and sensor data by the impact event monitoring device 102; capturing and recording the objects and the subjects and establishing communication between the impact event monitoring device 102 with the first computing device 106 and the second computing device 108 through the network module 218; reporting accurate positions and locations; the sensor data; media files; from the impact event monitoring device 102 to the first computing device 106 and the second computing device 108 and analyzing the accurate positions and locations; the sensor data; the media files of the objects by the impact event monitoring and analyzing module 114 for understanding the extent and severity of the impact events.

The second computing device 108 includes the impact data accessing module 116 may be configured to enable the second end-user to access the impact data and the media files of the impact event front and back with respect to time so that the second end-user may see the impact event from any angle. The media files may include, but not limited to, images, pictures, videos, GIFs, and so forth. The media files may be moved front and back with respect to time and the object/subject may be portrayed accordingly.

Referring to FIG. 2 is a block diagram 200 depicting the impact event monitoring device 102 shown in FIG. 1, in accordance with one or more exemplary embodiments. The impact event monitoring device 102 includes the processing device 203, a first set of sensors 204a, 204b and 204c, a second set of sensors 206a, 206b and 206c, a third set of sensors 208a, 208b, and 208c, an impact sensing unit 210, a motion detecting unit 212, a GPS module 214, an image capturing unit 216, and a network module 218, a memory unit 220, and a display unit 222. The first set of sensors 204a, 204b, 204c, the second set of sensors 206a, 206b and 206c, the third set of sensors 208a, 208b, and 208c may include, but not limited to, gyroscopes, accelerometers, compasses, pressure sensors, motion sensors, magnetometers, and so forth. The memory unit 220 may include the impact event monitoring and analyzing module 114.

The first set of sensors 204a, 204b and 204c may be electrically coupled to the processing device 203 and are configured to measure the linear acceleration and/or angular acceleration of the sensor array. The second set of sensors 206a, 206b and 206c may be electrically coupled to the processing device 203 and are configured to calibrate the exact orientations by measuring the Euler angles and/or quaternions. The third set of sensors 208a, 208b, and 208c may be electrically coupled to the processing device 203 and are configured to monitor vital statistics, the rotational angle of the head of the individual or the object at the time of the impact event. The third set of sensors 208a, 208b and 208c may also be configured to provide impact data to the second end-users to properly diagnose the extent and severity of the impact event. The impact sensing unit 210 may be electrically coupled to the processing device 203 and is configured to detect and determine the impact events that occur to the objects/subjects. The motion detecting unit 212 may be electrically coupled to the processing device 203 and is configured to measure changes in the orientation for having a continuous replication of the movement and/or motion of the objects/subjects.

The GPS module 214 may be electrically coupled to the processing device 203 and is configured to detect the accurate location of the impact events that occur to the objects/subjects. The image capturing unit 216 may be electrically coupled to the processing device 203 and is configured to record the video of the subjects and/or objects and capture the objects/subjects. For example, similar to live media, in a sense the image capturing unit 216 starts recording as soon as the first end-user opens the impact event monitoring and analyzing module 114 before the live media is captured. The live media may include, but not limited to, live photos, live media files, and so forth. The image capturing unit 216 may be configured to recreate the captured impact events (live media) in a 3D (three-dimensional) space.

The network module 218 may be electrically coupled to the processing device 203 and is configured to connect the impact event monitoring device 102 with the first computing device 106. The network module 218 may be configured to report the impact events as emergency notifications or impact notifications to the second computing device 108 over the network 110. The emergency notifications or the impact notifications may include but not limited to, SMS, alerts, email, warnings, and so forth. The network module 218 may also be configured to send a geographical location as a communication link and the information identifying the location of the objects and\or subjects to the second computing device 108 to communicate the portion of data stored in the memory unit 220. The information stored in the memory unit 220 may be preserved at least until an acknowledgment of receipt is received, representing successful transmission through the communication link. The memory unit 220 may be electrically coupled to the processing device 203 and is configured to receive movement or motion output and stores at least a portion of motion commencing at and/or before said determination. The display unit 222 may be electrically coupled to the processing device 203 and is configured to display the impact data, impact notifications, emergency notifications, and so forth.

The first set of sensors 204a, 204b, 204c, the second set of sensors 206a, 206b, and 206c and the third set of sensors 208a, 208b, and 208c may be configured to calculate the Pitch, Roll and Yaw data from the impact events. Pitch, Roll, and Yaw are the respective rotations of the object and/or the subject around the X, Y and Z axes respectively. The first set of sensors 204a, 204b, 204c, the second set of sensors 206a, 206b, and 206c and the third set of sensors 208a, 208b, and 208c may be configured to determine the mental state/consciousness/alertness of the first end-user post-crash or impact based on the analysis of motion sensors and data points plotted on X Y Z axis. The processing device 103 may be configured to obtain the values of Ax, Ay, Az, GYx, GYy and GYz from the impact sensing unit 210.

Pitch=180*a tan 2(Ax,Sqrt(Ay*Ay)+(Az*Az))/pi

Roll=180*a tan 2(Ay,Sqrt((Ax*Ax)+(Az*Az))/pi

Yaw=180*a tan 2(Az,Sqrt(Ax*Ax)+(Az*Az))/pi

Yaw value may change and is not the absolute value of it. Therefore, considering an assumed value initially, preferably 0. In order to calculate yaw, initially find the up direction using the accelerometer to measure g (gravity) (opposite to g is upwards) and using the gyroscope to measure the rate of turn on each axis. Scale these by the correct amount based on the orientation to obtain a yaw rate. Thereafter, integrating this yaw rate over time to obtain yaw values.

Tracking things while under significant motion is complex. Therefore, a pre-tuned KALMAN filter may be used to obtain accurate data. There is a buffer time of around 30-40 seconds after the impact event before initializing the analysis.

The impact event monitoring device 102 may be configured to calibrate the first set of sensors 204a, 204b, 204c, the second set of sensors 206a, 206b, 206c, and the third set of sensors 208a, 208b, and 208c. If the impact event is detected at a predetermined threshold value (for example, >70 g). The impact event monitoring device 102 may be configured to collect linear acceleration and angular acceleration at each point of impact event using the first set of sensors 204a, 204b, 204c, the second set of sensors 206a, 206b, 206c, and the third set of sensors 208a, 208b, and 208c. The impact event monitoring device 102 includes the processing device 103 may be configured to convert linear and angular acceleration data points into a function of time. The processing device 103 may be configured to convert linear and angular acceleration data points into yaw, pitch and roll data. The processing device 103 may be configured to track the head movement with yaw, pitch and roll data and analyse the yaw, pitch and roll data with machine learning algorithms for detecting level of consciousness and injuries that occur to objects, and the first end-user. The impact event monitoring device 102 may be configured activate quick emergency protocols and sending emergency notifications to the second computing device 108.

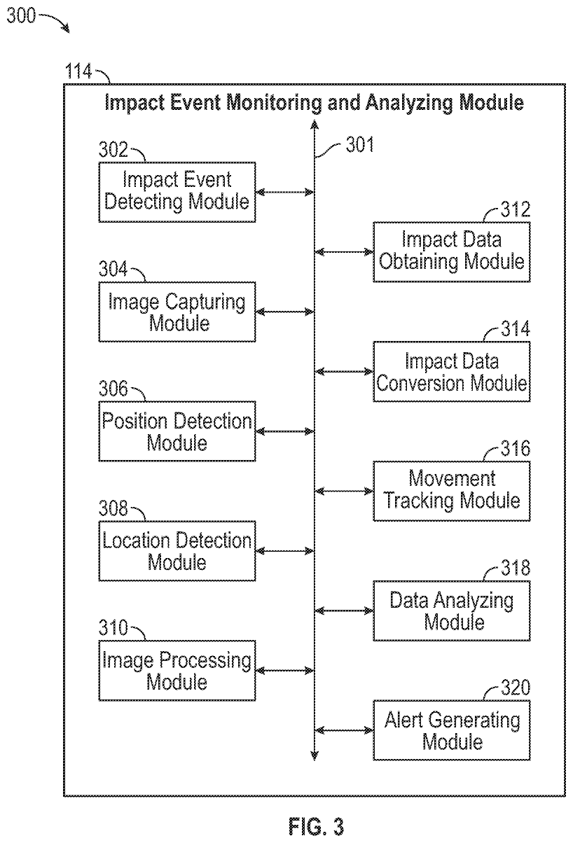

Referring to FIG. 3 is a block diagram 300 depicting a schematic representation of the impact event monitoring and analyzing module 114 shown in FIG. 1, in accordance with one or more exemplary embodiments. The impact event monitoring and analyzing module 114 includes a bus 301, an impact event detecting module 302, an image capturing module 304, a position detection module 306, a location detection module 308, and an image processing module 310, an impact data obtaining module 312, an impact data conversion module 314, a movement tracking module 316, and impact data analyzing module 318, and an alert generating module 320. The bus 301 may include a path that permits communication among the modules of the impact event monitoring and analyzing 114 installed on the computing devices 106, 108. The term “module” is used broadly herein and refers generally to a program resident in the memory of the computing devices 106, 108. The impact event monitoring and analyzing module 114 may include machine learning techniques, deep learning techniques, and computer-implemented pattern recognition techniques to detect anomalies or variations in normal behavior. The impact event detecting module 302 may be configured to detect the impact events that occur to the objects and/or the subjects.

The image capturing module 304 may be configured to capture the objects/subjects after experiencing the impact event. The image capturing module 304 may be configured to record the video of the subjects and/or objects and capture the objects/subjects at the time of impact event. The image capturing module 304 may be configured to recreate the captured impact events (live media) in a 3D (three-dimensional) space.

The position detection module 306 may be configured to fetch the object/subject positions (geo-location) “x” seconds before the impact interrupt and “x” seconds after the impact interrupt. The location detection module 308 may be configured to detect the accurate location of the impact events that occur to the objects/subjects. The image processing module 310 may be configured to convert the resulting “2x” seconds of the object/subject positions into a short animation/video by which the accurate object/subject positions at the time of the impact event may be reproduced.

The impact data obtaining module 312 may be configured to obtain the impact data from the image capturing module 304, the position detection module 306, the location detection module 308, and the image processing module 310. The impact data may include the accurate positions and locations and the sensor data. The accurate positions and locations may include, but not limited to, a head and/or body position of the subject, a geographical position of the object, a geographical position of the subject and so forth. The sensor data may be measured by the impact event monitoring device 102. The sensor data may include, but not limited to, quaternions, Euler angles, vital statistics, the rotational angle of the head of the individual or the object, movement and/or motion of the individual or the object, angular acceleration, linear acceleration, acceleration and gyroscope vectors, velocity, location, yaw, pitch and roll and so forth.

The impact data conversion module 314 may be configured to convert the impact data into a function of time. The impact data conversion module 314 may be configured to convert the impact data into yaw, pitch and roll data. The movement tracking module 316 may be configured to track the head movements with the yaw, pitch and roll data. The data analyzing module 318 may be configured to analyze the yaw, pitch and roll data with machine learning techniques and the deep learning techniques to detect consciousness level and injuries that occur to the objects and the subjects after experiencing the impact event. The data analyzing module 318 may be configured to analyze the yaw, pitch and roll data upon identifying no head movement and if the head is moving out of normal movement range.

The alert generating module 320 may be configured to generate the impact notifications/emergency notifications to the second computing device 108 to estimate the extent of the impact event by the second end-user. The impact notifications may be analyzed by the second end-user to understand the severity of the impact event. The alert generating module 320 may also be configured to deliver the impact data to medical examiners to properly diagnose the extent and severity of the injuries to provide better treatment for head/brain injuries.

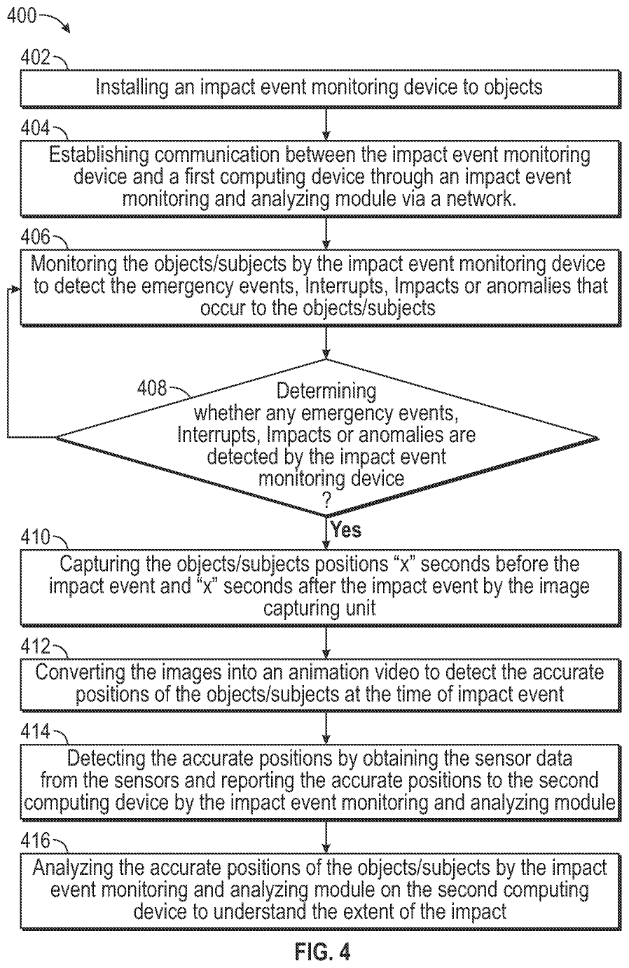

Referring to FIG. 4 is a flowchart 400 depicting an exemplary method of reporting impact events to the second end-users, in accordance with one or more exemplary embodiments. As an option, the exemplary method 400 is carried out in the context of the details of FIG. 1, FIG. 2 and FIG. 3. However, the exemplary method 400 is carried out in any desired environment. Further, the aforementioned definitions are equally applied to the description below.

The method commences at step 402, installing the impact event monitoring device to objects. Thereafter at step 404, establishing the communication between the impact event monitoring device and the first computing device through the impact event monitoring and analyzing module via the network. Thereafter at step 406, monitoring the objects/subjects by the impact event monitoring device to detect the impact events occur to the objects/subjects. Determining whether any impact events, interrupts, impacts or anomalies detected by the impact event monitoring device, at step 408. If the answer to step 408 is YES, the method continuous at step 410, capturing the objects/subjects positions “x” seconds before the impact event and “x” seconds after the impact event by the image capturing unit. Thereafter at step 412, converting the media files of objects/subjects into an animation video to detect the accurate positions and locations of the objects/subjects at the time of the impact event. Thereafter at step 414, detecting the accurate positions and locations by obtaining the additional sensor data from the sensors and reporting the accurate positions and locations to the second computing device through the impact event monitoring and analyzing module. Thereafter at step 416, analyzing the accurate positions and locations of the objects/subjects by the second end-users to understand the extent of the impact. If the answer to step 408 is NO, the method redirects at step 406.

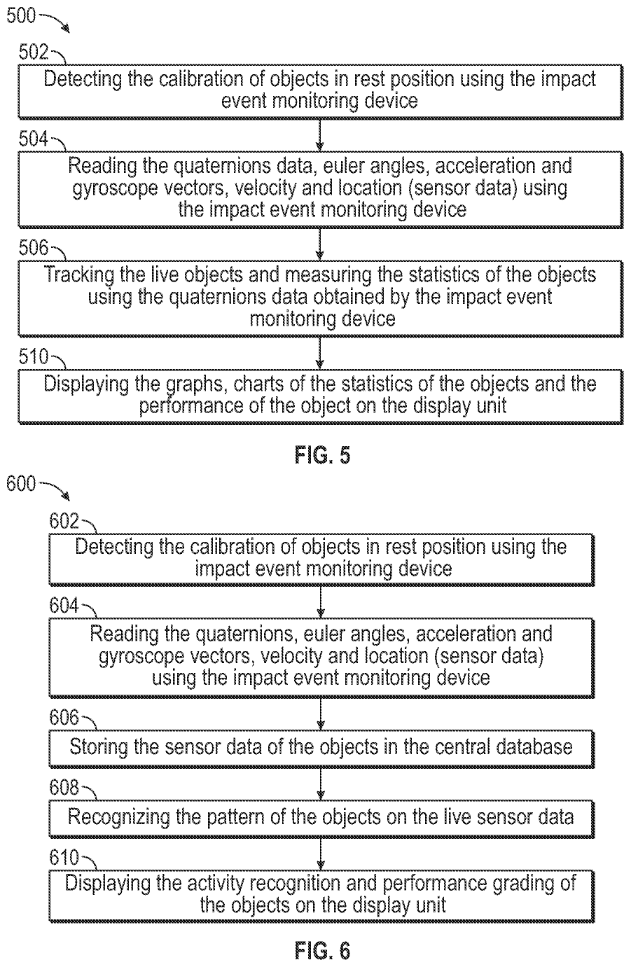

Referring to FIG. 5 is a flowchart 500 depicting an exemplary method of tracking the objects using the impact event monitoring device, in accordance with one or more exemplary embodiments. As an option, the exemplary method 500 is carried out in the context of the details of FIG. 1, FIG. 2, FIG. 3 and FIG. 4. However, the exemplary method 500 is carried out in any desired environment. Further, the aforementioned definitions are equally applied to the description below.

The method commences at step 502, detecting the calibration of objects in rest position using the impact event monitoring device. Thereafter at step 504, reading the quaternions data, Euler angles, acceleration and gyroscope vectors, velocity and location (sensor data) using the impact event monitoring device. Thereafter at step 506, tracking the live objects and measuring the statistics of the objects using the quaternions data obtained by the impact event monitoring device. Thereafter at step 508, displaying the graphs, charts of the statistics of the objects and the performance of the objects on the display unit.

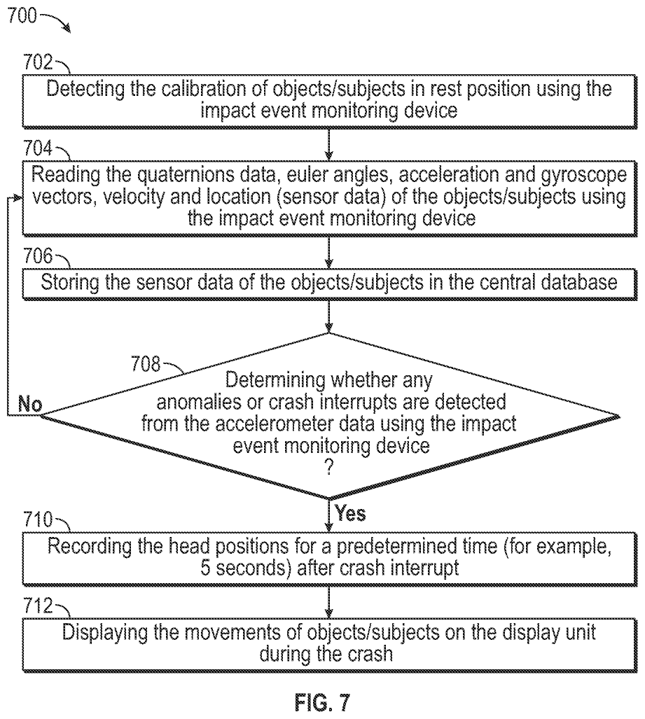

Referring to FIG. 6 is a flowchart 600 depicting an exemplary method of displaying the activity recognition and performance grading of the objects, in accordance with one or more exemplary embodiments. As an option, the exemplary method 600 is carried out in the context of the details of FIG. 1, FIG. 2, FIG. 3, FIG. 4, and FIG. 5. However, the exemplary method 600 is carried out in any desired environment. Further, the aforementioned definitions are equally applied to the description below.

The method commences at step 602, detecting the calibration of objects in rest position using the impact event monitoring device. Thereafter at step 604, reading the quaternions, Euler angles, acceleration and gyroscope vectors, velocity and location using the impact event monitoring device. Thereafter at step 606, storing the sensor data of the objects in the central database. Thereafter at step 608, recognizing the pattern of the objects on the live sensor data. Thereafter at step 610, displaying the activity recognition and performance grading of the objects on the display unit.

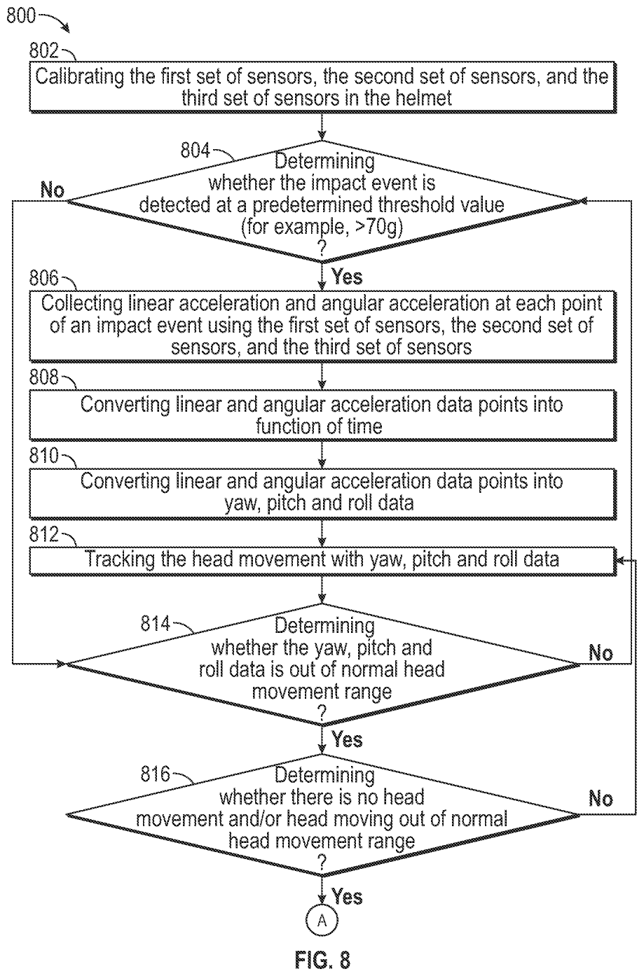

Referring to FIG. 7 is a flowchart 700 depicting an exemplary method of displaying the movements of objects/subjects during the crash, in accordance with one or more exemplary embodiments. As an option, the exemplary method 700 is carried out in the context of the details of FIG. 1, FIG. 2, FIG. 3 FIG. 4, FIG. 5, and FIG. 6. However, the exemplary method 700 is carried out in any desired environment. Further, the aforementioned definitions are equally applied to the description below.

The method commences at step 702, detecting the calibration of objects/subjects in rest position using the impact event monitoring device. Thereafter at step 704, reading the quaternions data, Euler angles, acceleration and gyroscope vectors, velocity and location (sensor data) of the objects/subjects using the impact event monitoring device. Thereafter at step 706, storing the sensor data of the objects/subjects in the central database. Determining whether any anomalies or crash interrupts are detected from the accelerometer data using the impact event monitoring device, at step 708. If the answer to step 708 is YES, the method continues at step 710, recording the head positions for a predetermined time (Fox example, 5 seconds) after crash interrupt. Thereafter at step 712, displaying the movements of objects/subjects on the display unit during the crash. If the answer at step 708 is NO, the method redirects at step 704.

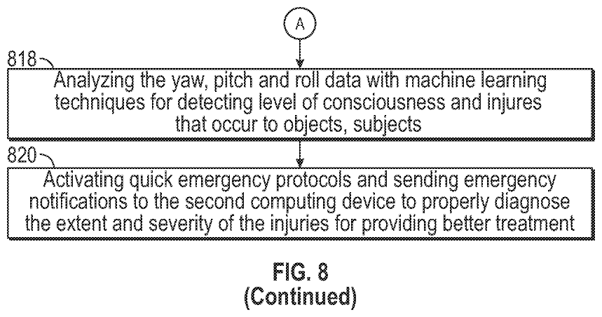

Referring to FIG. 8 is a flowchart 800 depicting an exemplary method for analyzing impact events and determining the consciousness of subjects after the crash, in accordance with one or more exemplary embodiments. As an option, the exemplary method 800 is carried out in the context of the details of FIG. 1, FIG. 2, FIG. 3, FIG. 4, FIG. 5, FIG. 6, and FIG. 7. However, the exemplary method 800 is carried out in any desired environment. Further, the aforementioned definitions are equally applied to the description below.

The method commences at step 802, calibrating the first set of sensors, the second set of sensors, and the third set of sensors in the helmet. Determining whether the impact event is detected at a predetermined threshold value (for example, >70 g)?, at step 804. If the answer at step 804 is Yes, the method continues at step 806, collecting linear acceleration and angular acceleration at each point of impact event using the first set of sensors, the second set of sensors, and the third set of sensors. Thereafter at step 808, converting linear and angular acceleration data points into a function of time. Thereafter at step 810, converting linear and angular acceleration data points into yaw, pitch and roll data. Thereafter at step 812, tracking the head movement with yaw, pitch and roll data. Determining whether the yaw, pitch and roll data is out of normal head movement range?, at step 814. If the answer at step 814 is Yes, the method continuous at step 816, Determining whether there is no head movement and/or head moving out of normal head movement range?. If the answer at step 816 is Yes, the method continues at step 818, analyzing the yaw, pitch and roll data with machine learning algorithms for detecting level of consciousness and injuries that occur to objects, and subjects. Thereafter at step 820, activating quick emergency protocols and sending emergency notifications to the second computing device. If the answer at step 804 is No, the method continues at step 814. If the answer at step 814 is No, the method reverts to step 804. If the answer at step 816 is No, the method reverts to step 812.

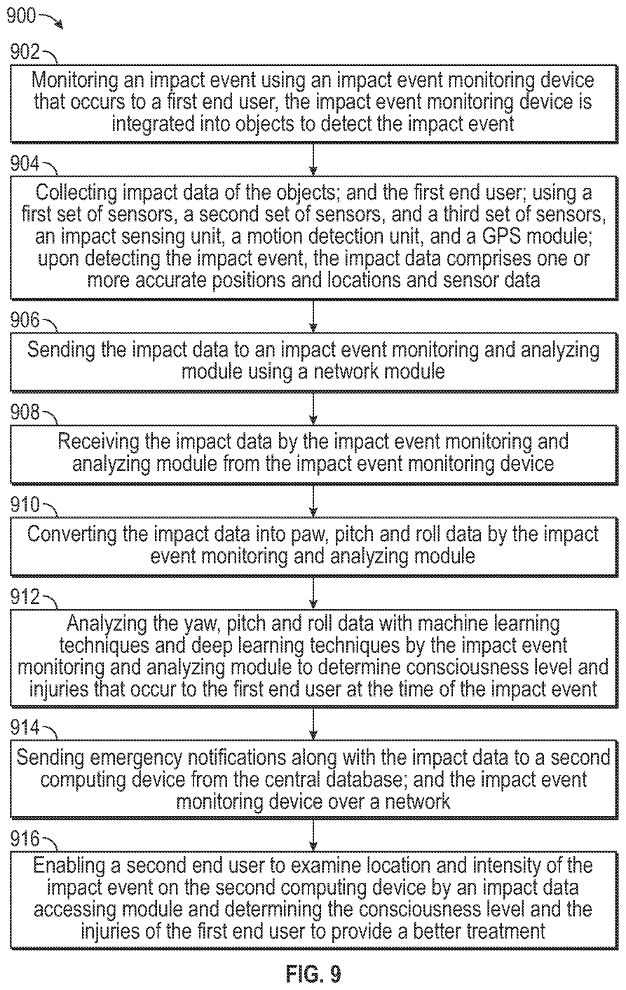

Referring to FIG. 9 is a flowchart 900 depicting another exemplary method for analyzing impact events and determining the consciousness of subjects after the crash, in accordance with one or more exemplary embodiments. As an option, the exemplary method 900 is carried out in the context of the details of FIG. 1, FIG. 2, FIG. 3, FIG. 4, FIG. 5, FIG. 6, FIG. 7, and FIG. 8. However, the exemplary method 900 is carried out in any desired environment. Further, the aforementioned definitions are equally applied to the description below.

The method commence at step 902, monitoring the impact event using the impact event monitoring device that occurs to the first end-user, the impact event monitoring device is integrated into objects to detect the impact event. Thereafter at step 904, collecting impact data of at least one of: the objects; and the first end-user; using a first set of sensors, a second set of sensors, and a third set of sensors, an impact sensing unit, a motion detection unit, and a GPS module; upon detecting the impact event, the impact data comprises one or more accurate positions and locations and sensor data. Thereafter at step 906, sending the impact data to the impact event monitoring and analyzing module using the network module. Thereafter at step 908, receiving the impact data by the impact event monitoring and analyzing module from the impact event monitoring device, the event monitoring and analyzing module is embedded in at least one of: the central database; the first computing device; and the impact event monitoring device. Thereafter at step 910, converting the impact data into Yaw, Pitch and Roll data by the impact event monitoring and analyzing module, the Yaw, Pitch and Roll data are respective rotations of the first end-user around X, Y and Z axes and the Yaw, Pitch and Roll data are used to track one or more head movements of the first end-user.

Thereafter at step 912, analyzing the Yaw, Pitch and Roll data with the machine learning techniques and one or more deep learning techniques by the impact event monitoring and analyzing module to determine the consciousness level and injuries that occur to the first end-user at the time of the impact event. Thereafter at step 914, sending the emergency notifications along with the impact data to a second computing device from at least one of the central database; and the impact event monitoring device over the network. Thereafter at step 916, enabling the second end-user to examine location and intensity of the impact event on the second computing device by the impact data accessing module, thereby determining the consciousness level and the injuries of the first end-user to provide better treatment.

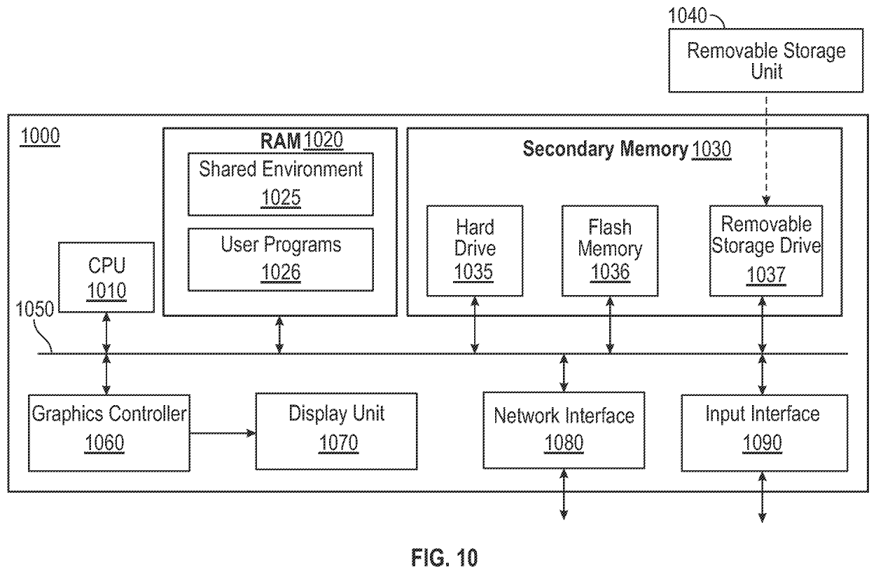

Referring to FIG. 10 is a block diagram 1000 illustrating the details of a digital processing system 1000 in which various aspects of the present disclosure are operative by execution of appropriate software instructions. The Digital processing system 1000 may correspond to the computing devices 106, 108 (or any other system in which the various features disclosed above can be implemented).

The digital processing system 1000 may contain one or more processors such as a central processing unit (CPU) 1010, random access memory (RAM) 1020, secondary memory 1027, graphics controller 1060, display unit 1070, network interface 1080, and input interface 1090. All the components except display unit 1070 may communicate with each other over communication path 1050, which may contain several buses as is well known in the relevant arts. The components of FIG. 10 are described below in further detail.

CPU 1010 may execute instructions stored in RAM 1020 to provide several features of the present disclosure. CPU 1010 may contain multiple processing units, with each processing unit potentially being designed for a specific task. Alternatively, CPU 1010 may contain only a single general-purpose processing unit.

RAM 1020 may receive instructions from secondary memory 1030 using communication path 1050. RAM 1020 is shown currently containing software instructions, such as those used in threads and stacks, constituting shared environment 1025 and/or user programs 1026. Shared environment 1025 includes operating systems, device drivers, virtual machines, etc., which provide a (common) run time environment for execution of user programs 1026.

Graphics controller 1060 generates display signals (e.g., in RGB format) to display unit 1070 based on data/instructions received from CPU 1010. Display unit 1070 contains a display screen to display the images defined by the display signals. Input interface 1090 may correspond to a keyboard and a pointing device (e.g., touch-pad, mouse) and may be used to provide inputs. Network interface 1080 provides connectivity to a network (e.g., using Internet Protocol), and may be used to communicate with other systems (such as those shown in FIG. 1) connected to the network 110.

Secondary memory 1030 may contain hard drive 1035, flash memory 1036, and removable storage drive 1037. Secondary memory 1030 may store the data software instructions (e.g., for performing the actions noted above with respect to the Figures), which enables digital processing system 1000 to provide several features in accordance with the present disclosure.

Some or all of the data and instructions may be provided on removable storage unit 1040, and the data and instructions may be read and provided by removable storage drive 1037 to CPU 1010. Floppy drive, magnetic tape drive, CD-ROM drive, DVD Drive, Flash memory, removable memory chip (PCMCIA Card, EEPROM) are examples of such removable storage drive 1037.

Removable storage unit 1040 may be implemented using medium and storage format compatible with removable storage drive 1037 such that removable storage drive 1037 can read the data and instructions. Thus, removable storage unit 1040 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 1040 or hard disk installed in hard drive 1035. These computer program products are means for providing software to digital processing system 1000. CPU 1010 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 1030. Volatile media includes dynamic memory, such as RAM 1020. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (communication path) 1050. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

Although the present disclosure has been described in terms of certain preferred embodiments and illustrations thereof, other embodiments and modifications to preferred embodiments may be possible that are within the principles of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

In an embodiment of the present disclosure, a system for monitoring and analyzing impact data to determine consciousness level and injuries, includes an impact event monitoring device 102 configured to monitor an impact event that occurs to a first end-user, the impact event monitoring device 102 is integrated into objects to detect the impact event.

In another embodiment of the present disclosure, the impact event monitoring device 102 includes a processing device 103 configured to collect impact data of the objects; and/or the first end-user using a first set of sensors 204a, 204b, 204c, a second set of sensors 206a, 206b, 206c, and a third set of sensors 208a, 208b, 208c, an impact sensing unit 210, a motion detection unit 212, a GPS module 214, and an image capturing unit 216 upon detecting the impact event, the impact data may include accurate positions and locations and sensor data.

In another embodiment of the present disclosure, a network module 218 configured to send the impact data to an impact event monitoring and analyzing module 114, the impact event monitoring and analyzing module 114 configured to receive impact data from the impact event monitoring device 102 and convert the impact data into Yaw, Pitch and Roll data, the Yaw, Pitch and Roll data are respective rotations of the first end-user around X, Y and Z axes and the Yaw, Pitch and Roll data are used to track one or more head movements of the first end-user.

In another embodiment of the present disclosure, the impact event monitoring and analyzing module 114 also configured to analyze the Yaw, Pitch and Roll data with machine learning techniques and deep learning techniques to determine consciousness level and injuries that occur to the first end-user in the impact event.

In another embodiment of the present disclosure, the impact event monitoring and analyzing module 114 is configured to send emergency notifications along with the impact data to a second computing device 108 over a network 110, the second computing device 108 includes an impact data accessing module 116 configured to enable a second end-user to examine location and intensity of the impact event to understand the consciousness level and the injuries for providing better treatment to the first end-user.

In another embodiment of the present disclosure, the accurate positions and locations comprises a head position, and body position of the first end-user, a geographical position of the object, a geographical position of the first end-user. The sensor data comprises quaternions, Euler angles, vital statistics, the rotational angle of the head of the first end-user, the rotational angle of the object, movement and/or motion of the first end-user or the object, an angular acceleration, a linear acceleration, acceleration and gyroscope vectors, and velocity. The impact event monitoring and analyzing module 114 is embedded in a central database 112 and is configured to collect impact data from at least one of: the impact event monitoring device 102, and the first computing device 106.

In another embodiment of the present disclosure, the impact event monitoring and analyzing module 114 is configured to collect the impact data from the impact event monitoring device 102 over the network 110. The impact event monitoring and analyzing module 114 is configured to collect the impact data from the impact event monitoring device 102 in combination with the first computing device 106. The impact event monitoring and analyzing module 114 comprises an impact event detecting module 302 is configured to detect the impact event that occurs to at least one of: the objects; and the first end-user.

In another embodiment of the present disclosure, an image capturing module 304 is configured to capture at least one of: the objects; and the first end-user after experiencing the impact event, a position detection module 306 is configured to fetch at least one of: head positions; and body positions; of the first end-user after experiencing the impact event, a location detection module 308 is configured to fetch at least one of: a geographical location of the objects; and a geographical location of the first end-user at the time of the impact event, an image processing module 310 is configured to convert the resulting “2x” seconds of at least one of: objects position; and the first end-user positions into a short animation video by which the accurate positions of at least one of: the objects; and the first end-user at the time of the impact event is reproduced, an impact data obtaining module 312 is configured to obtain the impact data from the image capturing module, the position detection module, the location detection module, and the image processing module, an impact data conversion module 314 is configured to convert the impact data into function of time, the impact data conversion module 314 is configured to convert the impact data into Yaw, Pitch and Roll data, a movement tracking module 316 is configured to track one or more head movements with the Yaw, Pitch and Roll data, a data analyzing module 318 is configured to analyze the Yaw, Pitch and Roll data with one or more machine leaning techniques and deep learning techniques to detect the consciousness level and injuries that occur to the first end-user at the time of the impact event, the data analyzing module 318 is configured to analyze the Yaw, Pitch and Roll data upon identifying no head movements and if the head is moving out of normal movement range, an alert generating module 320 is configured to deliver the emergency notifications to the second computing device 108 for enabling the second end-user to estimate the intensity of the impact event to provide better treatment.

In another embodiment of the present disclosure, the second computing device 108 includes the impact data accessing module 116 is configured to enable the second end-user to move media files of the impact event front and back with respect to time and to view the impact event from any angle.

In another embodiment of the present disclosure, the first set of sensors 204a, 204b, and 204c configured to measure a linear acceleration, a linear velocity, an angular acceleration, jerks, quaternions, Euler angles, vital statistics, a rotational angle, a geographical location, movement and/or motion and gyroscope vectors of at least one of: the objects; and the first end-user, the second set of sensors 206a, 206b, 206c configured to calibrate one or more orientations of at least one of: the objects; and the first end-user; by measuring Euler angles and/or quaternions, the third set of sensors configured to monitor one or more vital statistics, rotational angle of the head of the first end-user at the time of the impact event, the third set of sensors 208a, 208b, 208c are configured to provide an additional sensor data to the second computing device for proper diagnosing extent and severity of the impact event.

In another embodiment of the present disclosure, the impact sensing unit 210 configured to detect and determine the impact event that occurs to at least one of: the objects; and the first end-user, the motion detecting unit 212 configured to measure changes in the one or more orientations for continuous replication of a movement and/or motion of at least one of: the objects; and the first end-user, the GPS module 214 configured to detect an accurate location at the time of the impact event that occurs to at least one of: the objects; the first end-user, the network module 218 is configured to establish communication between the impact event monitoring device 102 and at least one of: the first computing device 106; the second computing device 108; to send one or more emergency notifications of the impact event, the network module 218 is configured to send the geographical location as a communication link, and an information identifying the location of at least one of: the objects; and the first end-user; to the second computing device 108 for communicating the impact data stored in a memory unit 220 of the impact event monitoring device 102.

In an embodiment of the present disclosure, a method for monitoring and analyzing impact data to determine consciousness level and injuries, comprising: monitoring an impact event using an impact event monitoring device 102 that occurs to a first end-user, the impact event monitoring device 102 is integrated into objects to detect the impact event; collecting impact data of at least one of: the objects; and the first end-user; using a first set of sensors 204a, 204b, 204c, a second set of sensors 206a, 206b, 206c, and a third set of sensors 208a, 208b, 208c, an impact sensing unit 210, a motion detection unit 212, a GPS module 214 and an image capturing unit 216; upon detecting the impact event, the impact data comprises one or more accurate positions and locations and sensor data; sending the impact data to an impact event monitoring and analyzing module 114 using a network module 218; receiving the impact data by the impact event monitoring and analyzing module 114 from the impact event monitoring device 102, the event monitoring and analyzing module 114 is embedded in at least one of: a central database 112; a first computing device 106; and the impact event monitoring device 102; converting the impact data into Yaw, Pitch and Roll data by the impact event monitoring and analyzing module 114, the Yaw, Pitch and Roll data are respective rotations of the first end-user around X, Y and Z axes and the Yaw, Pitch and Roll data are used to track one or more head movements of the first end-user; analyzing the Yaw, Pitch and Roll data with one or more machine learning techniques and one or more deep learning techniques by the impact event monitoring and analyzing module 114 to determine consciousness level and injuries that occur to the first end-user at the time of the impact event; reporting and sending emergency notifications along with the impact data to a second computing device 108 from at least one of the central database 112; and the impact event monitoring device 102 over a network 110; and enabling a second end-user to examine location and intensity of the impact event on the second computing device 108 by an impact data accessing module 116 and determining the consciousness level and the injuries of the first end-user to provide a better treatment.

In another embodiment of the present disclosure, collecting the impact data by the impact event monitoring and analyzing module 114 from the impact event monitoring device 102 in combination with the first computing device 106; converting the impact data into a function of time by an impact data conversion module 314; tracking the one or more head movements with the Yaw, Pitch and Roll data by a movement tracking module 316; enabling the second end-user to access media files of the impact event front and back with respect to time and to view the impact event from any angle by the impact data accessing module 116 on the second computing device 108.

What is claimed is:

1. A system for monitoring and analyzing impact data to determine consciousness level and injuries, comprising:
an impact event monitoring device configured to monitor an impact event that occurs to a first end-user, whereby the impact event monitoring device is integrated into one or more objects to detect the impact event;
the impact event monitoring device comprises a processing device configured to collect impact data of at least one of: the objects; and the first end-user; using a first set of sensors, a second set of sensors, and a third set of sensors, an impact sensing unit, a motion detection unit, a GPS module, and an image capturing unit upon detecting the impact event, the impact data comprises one or more accurate positions and locations and sensor data;
a network module configured to send the impact data to an impact event monitoring and analyzing module, wherein the impact event monitoring and analyzing module configured to receive impact data from the impact event monitoring device and convert the impact data into Yaw, Pitch and Roll data, wherein the Yaw, Pitch and Roll data are respective rotations of the first end-user around X, Y and Z axes and the Yaw, Pitch and Roll data are used to track one or more head movements of the first end-user, whereby the impact event monitoring and analyzing module also configured to analyze the Yaw, Pitch and Roll data with one or more machine learning techniques and one or more deep learning techniques to determine consciousness level and one or more injuries that occur to the first end-user in the impact event; and
the impact event monitoring and analyzing module configured to report and send emergency notifications along with the impact data to a second computing device over a network, whereby the second computing device comprises an impact data accessing module configured to enable a second end-user to examine location and intensity of the impact event to understand the consciousness level and the injuries for providing better treatment to the first end-user.

2. The system of claim 1, wherein the accurate positions and locations comprise a head position, and body position of the first end-user, a geographical position of the object, a geographical position of the first end-user.

3. The system of claim 1, wherein the sensor data comprises quaternions, Euler angles, vital statistics, the rotational angle of the head of the individual, the rotational angle of the object, movement and/or motion of the first end-user or the object, an angular acceleration, an linear acceleration, acceleration and gyroscope vectors, and velocity.

4. The system of claim 1, wherein the impact event monitoring and analyzing module is configured to collect the impact data from the impact event monitoring device over a network.

5. The system of claim 1, wherein the impact event monitoring and analyzing module is configured to collect the impact data from the impact event monitoring device in combination with the first computing device.

6. The system of claim 1, wherein the impact event monitoring and analyzing module comprises an impact event detecting module is configured to detect the impact event that occurs to at least one of: the objects; and the first end-user.

7. The system of claim 1, wherein the impact event monitoring and analyzing module comprises an image capturing module is configured to capture at least one of: the objects; and the first end-user after experiencing the impact event.

8. The system of claim 1, wherein the impact event monitoring and analyzing module comprises a position detection module is configured to fetch at least one of: head positions; and body positions; of the first end-user after experiencing the impact event.

9. The system of claim 1, wherein the impact event monitoring and analyzing module comprises a location detection module configured to fetch at least one of: a geographical location of the objects; and a geographical location of the first end-user at the time of the impact event.

10. The system of claim 1, wherein the impact event monitoring and analyzing module comprises an impact data conversion module is configured to convert the impact data into function of time.

11. The system of claim 1, wherein the impact event monitoring and analyzing module comprises a data analyzing module is configured to analyze the Yaw, Pitch and Roll data with one or more machine leaning techniques and deep learning techniques to detect the consciousness level and injuries that occur to the first end-user at the time of the impact event.

12. The system of claim 11, wherein the data analyzing module is configured to analyze the Yaw, Pitch and Roll data upon identifying no head movements and if the head is moving out of normal movement range.

13. The system of claim 1, wherein the impact event monitoring and analyzing module comprises an alert generating module configured to deliver the one or more emergency notifications to the second computing device for enabling the second end-user to estimate the intensity of the impact event to provide better treatment.

14. The system of claim 1, wherein the one or more first set of sensors configured to measure a linear acceleration, a linear velocity, an angular acceleration, jerks, quaternions, Euler angles, vital statistics, a rotational angle, a geographical location, movement and/or motion and gyroscope vectors of at least one of: the objects; and the first end-user.

15. The system of claim 1, wherein the one or more second set of sensors configured to calibrate one or more orientations of at least one of: the objects; and the first end-user; by measuring Euler angles and/or quaternions.

16. The system of claim 1, wherein the one or more third set of sensors configured to monitor one or more vital statistics, rotational angle of the head of the first end-user at the time of the impact event.

17. The system of claim 1, wherein the motion detecting unit is configured to measure changes in the one or more orientations for continuous replication of a movement and/or motion of at least one of: the objects; and the first end-user.

18. The system of claim 1, wherein the GPS module is configured to detect an accurate location at the time of the impact event that occurs to at least one of: the objects; the first end-user.

19. The system of claim 1, wherein the network module is configured to send the geographical location as a communication link, and an information identifying the location of at least one of: the objects; and the first end-user; to the second computing device for communicating the impact data stored in a memory unit of the impact event monitoring device.

20. A method for monitoring and analyzing impact data to determine consciousness level and injuries, comprising:
monitoring an impact event using an impact event monitoring device that occurs to a first end-user, whereby the impact event monitoring device is integrated into one or more objects to detect the impact event;
collecting impact data of at least one of: the objects; and the first end-user; using a first set of sensors, a second set of sensors, and a third set of sensors, an impact sensing unit, a motion detection unit, a GPS module, and an image capturing unit; upon detecting the impact event, whereby the impact data comprises one or more accurate positions and locations and sensor data;
sending the impact data to an impact event monitoring and analyzing module using a network module;
receiving the impact data by the impact event monitoring and analyzing module from the impact event monitoring device, whereby the impact event monitoring and analyzing module is embedded in at least one of: a central database; a first computing device; and the impact event monitoring device;
converting the impact data into Yaw, Pitch and Roll data by the impact event monitoring and analyzing module, wherein the Yaw, Pitch and Roll data are respective rotations of the first end-user around X, Y and Z axes and the Yaw, Pitch and Roll data are used to track one or more head movements of the first end-user;
analyzing the Yaw, Pitch and Roll data with one or more machine learning techniques and one or more deep learning techniques by the impact event monitoring and analyzing module to determine consciousness level and injuries that occur to the first end-user at the time of the impact event; and
sending emergency notifications along with the impact data to a second computing device from at least one of the central database; and the impact event monitoring device over a network; and
enabling a second end-user to examine location and intensity of the impact event on the second computing device and determining the consciousness level and the injuries of the first end-user to provide better treatment.
